(19) United States
(12) Patent Application Publication
(10) Pub. No.: US 2025/0281461 A1
(43) Pub. Date: Sep. 11, 2025

(54) USE OF MEBENDAZOLE FOR TREATING VIRAL INFECTION

(71) Applicants: The Board of Supervisors of Louisiana State University and Agricultural and Mechanical College, Baton Rouge, LA (US); Skymount Medical US Inc., New Orleans, LA (US)

(72) Inventors: Adam BESS, Baton Rouge, LA (US); Frej Knut Gosta BERGLIND, Baton Rouge, LA (US); Supratik MUKHOPADHYAY, Baton Rouge, LA (US); Kishor M. WASAN, Saskatoon, Saskatchewan (CA); Chris GALLIANO, New Orleans, LA (US); Michal BRYLINSKI, Baton Rouge, LA (US); Stephania CORMIER, Baton Rouge, LA (US); Allan ADER, New Orleans, LA (US); Nicholas GRIGGS, New Orleans, LA (US); Janet GOULD, New Orleans, LA (US); Tiffany CHO, New Orleans, LA (US); Julia ABRAMOV, New Orleans, LA (US); Peter HNIK, New Orleans, LA (US)

(73) Assignees: Board of Supervisors of Louisiana State University and Agricultural and Mechanical College, Baton Rouge, LA (US); Skymount Medical US Inc., New Orleans, LA (US)

(21) Appl. No.: 18/283,704

(22) PCT Filed: Mar. 23, 2022
(86) PCT No.: PCT/US22/21598

§ 371 Date: Sep. 22, 2023

Related U.S. Application Data
(60) Provisional application No. 63/165,510, filed on Mar. 24, 2021.

(51) Int. Cl. A61K 31/4184; A61K 31/506; A61P 31/14

(52) U.S. Cl. CPC A61K 31/4184; A61K 31/506; A61P 31/14

(57) ABSTRACT

Embodiments of the present disclosure relate to use of Mebendazole, a tyrosine kinase inhibitor or both in combination to treat a coronavirus infection in a subject.

### TECHNICAL FIELD

Embodiments of the present disclosure relate to use of compounds and compositions for treating a viral infection. In particular, the embodiments of the present disclosure relate to use of compounds and compositions for treating a coronavirus infection.

### BACKGROUND

The appearance of a novel coronavirus, referred to as SARS-COV-2, on the world stage has affected substantially every population in the world. This virus has afflicted millions of individuals and caused a disease, referred to as COVID-19. COVID-19 can develop into a significant health risk and result in death, which has placed a high strain on healthcare resources and society in general.

SARS-COV-2 is a single-strand, positive-sense ribonucleic acid (RNA) virus with a similar receptor-binding domain structure to that of SARS-COV and MERS-COV. SARS-COV-2 is transmitted between individuals via airborne droplets accessing nasal mucosa. Within the nasal mucosa SARS-COV-2 can rapidly reproduce and be shed in nasal secretions (sputum). Sputum can be transmitted to other individuals via airborne droplets, thus repeating the transmission cycle. The SARS-COV-2 virus can spread between individuals before the onset of symptoms, during the symptomatic period and even after recovery.

The clinical spectrum of the infection is wide, ranging from mild signs of an upper respiratory tract infection to severe pneumonia, multi-organ failure and death. At the onset, SARS-COV-2 primarily attacks the respiratory system, as it represents the main point of entry into the host, but SARS-CoV-2 also can affect multiple organs of an infected individual. The severity of COVID-19 is typically associated with comorbidities such as, but not limited to: hypertension, diabetes, obesity, and/or advanced age that can exacerbate the consequences of COVID-19.

There exists a need for a therapy that is capable of mitigating the impact of COVID-19 in such a manner that it slows down physiological impact on an infected individual.

### SUMMARY

Embodiments of the present disclosure provide one or more therapies for ameliorating and/or inhibiting some or substantially all of the risks, symptoms and development of severe disease in a subject infected with a coronavirus. In some embodiments of the present disclosure, the coronavirus is SARS-COV-2.

Some embodiments of the present disclosure relate to a use of Mebendazole for ameliorating and/or inhibiting some or substantially all of the risks, symptoms and development of severe disease caused by a coronavirus infection.

Some embodiments of the present disclosure relate to a method of treating an individual infected with a coronavirus, wherein said method comprises the steps of: providing a therapeutically effective amount of Mebendazole; and, administering the therapeutically effective amount of Mebendazole to said individual to ameliorate and/or inhibit some or substantially all of the risks, symptoms and development of severe disease in a subject infected with a coronavirus.

Some embodiments of the present disclosure relate to a use of Mebendazole and a tyrosine kinase inhibitor for ameliorating and/or inhibiting some or substantially all of the risks, symptoms or development of severe disease caused by a coronavirus infection.

Some embodiments of the present disclosure relate to a method of treating an individual infected with a coronavirus, wherein said method comprises the steps of: providing a therapeutically effective amount of Mebendazole and a therapeutically effective amount of a tyrosine kinase inhibitor; administering said therapeutically effective amounts of Mebendazole and the tyrosine kinase inhibitor to said individual for ameliorating and/or inhibiting some or substantially all of the risks, symptoms or development of severe disease caused by a coronavirus infection.

Some embodiments of the present disclosure relate to a method of making an agent/target cell complex, the method comprising a step of administering a therapeutically effective amount of the agent to a subject, wherein the agent/target cell complex inhibits a virus from entering, fusing with and/or replicating within the cells of the agent/target complex. In some embodiments of the present disclosure, the agent is Mebendazole or a tyrosine kinase inhibitor or both.

Some embodiments of the present disclosure relate to a method of making an agent/target virion complex, the method comprising a step of administering a therapeutically effective amount of the agent to a subject, wherein the agent/target virion complex inhibits the agent/target virion complex from entering, fusing with and/or replicating within one or more cells of a subject. In some embodiments of the present disclosure, the agent is Mebendazole or a tyrosine kinase inhibitor or both.

Some embodiments of the present disclosure relate to a pharmaceutical composition that comprises Mebendazole in a first therapeutically effective amount; a tyrosine kinase inhibitor in a second therapeutically effective amount, wherein the first and second therapeutically effective amounts are different or not; and at least one excipient.

Without being bound by any particular theory, it is postulated that Mebendazole may target the SARS-COV-2 virus by inhibiting the entry or fusion of the virus with a subject's cell and/or inhibiting replication of the virus once inside a subject's cell. While Mebendazole is a known anthelminthic, surprisingly it may also be useful in treating COVID-19 because Mebendazole interferes in viral tubulin formation and it may target viral calmodulin-domain protein kinase 1 also.

Without being bound by any particular theory, it is postulated that a tyrosine kinase inhibitor, such as Imatinib, when used in combination with Mebendazole, may also be useful as a therapy for ameliorating and/or inhibiting some or substantially all of the risks, symptoms and development of severe disease in a subject infected with a coronavirus. In some embodiments of the present disclosure, Imatinib which is a known ABL kinase inhibitor, may be useful in as part of a combination treatment, with Mebendazole, for COVID-19 because Imatinib is known to target one or more of TK ALK, platelet-derived growth factor receptor alpha, TK ABL1/Bcr-Abl, or Mast/stem cell growth factor receptor Kit.

### BRIEF DESCRIPTION OF THE DRAWINGS

These and other features of the present disclosure will become more apparent in the following detailed description in which reference is made to the appended drawings.

FIG. 1 shows in vitro experimental data regarding the percent viral inhibition and percent inhibition of cell viability cells treated with Mebendazole.

FIG. 2 shows in vitro experimental data from cells treated with Imatinib, wherein FIG. 2A shows TCID50/mL data (as a percent of control); and, wherein FIG. 2B shows the percent inhibition data.

FIG. 3 shows in vitro cytotoxicity experimental data from two cell lines treated with Imatinib.

FIG. 4 shows in vitro percent viral inhibition experimental data and percent cytotoxicity experimental data from cells treated with Mebendazole and Imatinib.

FIG. 5 is a Kaplan-Meier survival curve showing in vivo experimental data from mice infected with SARS-COV-2 virus and then treated with a placebo or various treatments according to embodiments of the present disclosure.

FIG. 6 is a line graph show the percent body weight change in the mice of FIG. 5.

FIG. 7 is a histogram showing in vivo experimental data from the mice of FIG. 5, including serum levels of ALT, AST and BUN from the control group and the groups of mice treated according to embodiments of the present disclosure.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of a biomolecule.

As used herein, the terms “agent” and “therapeutic agent” refer to a substance that, when administered to a subject, causes one or more chemical reactions and/or one or more physical reactions and/or or one or more physiological reactions and/or one or more pharmacological reactions and/or one or more immunological reactions in the subject.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target virions. This association results in a change in the metabolism or functionality of the target virions. As used herein, the phrase “change in metabolism” refers to an increase or a decrease in the one or more target virions' production of one or more proteins, and/or any post-translational modifications of one or more proteins. As used herein, the phrase “change in functionality” refers to a difference in physiological function of one or more aspects of a virion within an agent/virion complex as compared to a virion that is not part of such a complex.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “excipient” refers to any substance, not itself an agent, which may be used in a composition for delivery of one or more agents, and the like to a subject or alternatively combined with . . . one or more carriers and the like (e.g., to create a pharmaceutical composition) to improve its handling or storage properties or to permit or facilitate formation of a dose unit of the composition (e.g., formation of a topical hydrogel which may then be optionally incorporated into a transdermal patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form the pharmaceutical compositions. Any such excipients can be used in any dosage forms according to the present disclosure. The foregoing classes of excipients are not meant to be exhaustive but merely illustrative.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises an agent and that can promote recovery from a disease, disorder or symptom thereof and/or that can prevent a disease, disorder or symptom thereof and/or that can inhibit the progression of a disease, disorder, or symptom thereof.

As used herein, the term “pharmaceutical composition” means any composition comprising, but not necessarily limited to, one or more agents to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the one or more agents. Some examples of pharmaceutically acceptable carriers and their formulations are described in Remington (1995, The Science and Practice of Pharmacy (19th ed.) ed. A. R. Gennaro, Mack Publishing Company, Easton, PA), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2,3-dioleyloxy) propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation suits the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevent”, “prevention of” and “preventing” refer to avoiding the onset or progression of a disease, disorder, or a symptom thereof.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cell types within a subject that can interact with a coronavirus by the virus fusing with the outer membrane of the one or more cell types, entering into the cell and/or replicating therein. Without being bound to any particular theory, target cells of a subject can include any cells within a subject that express the receptors and/or co-factors required for viral interaction. Examples of these types of cells include, but are not limited to: epithelial cells of the upper airways and conducting airways (ciliated and non-ciliated); alveolar epithelial cells (both type 1 and 2); epithelial cells and neurons of the olfactory system; neurons of the central or peripheral nervous system; epithelial cells, enterocytes and gland cells of the gastrointestinal tract; cells of the blood, including immune effector cells; cardiovascular cells; and, renal cells.

As used herein, the term “target virion” refers to one or more viral particles of coronavirus that have the capacity to cause a viral infection within a subject cell. In some embodiments of the present disclosure, the viral particles are of one or more variants of SARS-COV-2.

As used herein, the term “therapeutically effective amount” refers to the amount of the agent used that is of sufficient quantity to ameliorate, prevent, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and, (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise one or more agents that may be administered to a subject by one or more routes, such as oral, intra-venous injection, intra-muscular injection, topical, transmucosal or combinations thereof. In some embodiments of the present disclosure, the one or more agents are administered orally to deliver a therapeutically effective amount, characterized as a total predetermined daily dose that is administered over a course of a predetermined number of days. In some embodiments of the present disclosure, the total predetermined daily dose may be administered in one or more smaller doses through a given day so that when all smaller doses are administered, the subject will have received the total predetermined daily dose.

In embodiments of the present disclosure, the pharmaceutical compositions disclosed herein comprise one or more agents, as described herein, in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

In embodiments of the present disclosure, the pharmaceutical compositions disclosed herein comprise two or more agents as described above in a total amount by weight of the composition of about 0.1% to about 95%. For example, the amount of a first agent and a second agent may be the same or different. The amount of the first agent and the second agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

In embodiments of the present disclosure, the pharmaceutical compositions disclosed herein comprise two or more agents as described above in a total amount by mass of each agent, whereby a subject may be administered a given mass of each of the two or more agents so as to receive the predetermined daily amount. For example, the amount of a first agent and a second agent may be the same or different. In some embodiments of the present disclosure, predetermined daily amount of the first agent may be between about 25 mg/day and about 500 mg/day, or between about 50 mg/day and about 450 mg/day, or between about 75 mg/day and about 425 mg/day, or between about 100 mg/day and about 400 mg/day, or between about 125 mg/day and about 375 mg/day, or between about 150 mg/day and about 350 mg/day, or between about 175 mg/day and about 325 mg/day, or between about 200 mg/day and about 300 mg/day. In some embodiments of the present disclosure, the predetermined daily dose of the first agent may be between about 185 mg/day and about 215 mg/day, or between about 190 mg/day and about 210 mg/day, or between about 195 mg/day and about 205 mg/day. In some embodiments of the present disclosure, the predetermined daily dose of the first agent may be about 200 mg/day, delivered orally and the first agent may be Mebendazole.

In some embodiments of the present disclosure predetermined daily dose of the second agent may be zero or it may be between about 400 mg/day and about 1200 mg/day, or between about 500 mg/day and about 1100 mg/day, or between about 600 mg/day and about 1000 mg/day, or between about 700 mg/day and about 900 mg/day. In some embodiments of the present disclosure, the predetermined daily dose of the second agent may be between about 700 mg/day and about 900 mg/day, or between about 725 mg/day and about 875 mg/day, or between about 750 mg/day and about 850 mg/day, or between about 775 mg/day and about 825 mg/day. In some embodiments of the present disclosure, the predetermined daily dose of the second agent may be about 800 mg/day, delivered orally and the second agent may be a tyrosine kinase inhibitor, such as Imatinib.

In some embodiments of the present disclosure, the first agent and the second agent may be administered separately or they may be administered as a single combined therapy, over a course of between 5 and 15 days, or between 6 and 14 days, or between 7 and 13 days, or between 8 and 12 days, or between 9 and 11 days. In some embodiments of the present disclosure, the subject is administered the predetermined daily dose of the first agent and the predetermined daily dose of the second agent over a course of about 10 days. In the embodiments where the first and second agents are administered separately, both agents will be given with at least 60-minute interval (starting with Imatinib followed by Mebendazole 60+ minutes later).

In the embodiments where the first and second agents are administered as a single combined therapy, the amount of the first agent and the second agent within a given administration of the combined therapy may provide the entire predetermined daily dose of the first and second agent, or a portion of each agent's predetermined daily dose.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

COVID-19 patients have demonstrated that the time from symptom onset to development of dyspnea may be between 5 to 10 days. In some COVID-19 patients, it may take between 10 and 14 days to develop severe respiratory distress syndrome. The probability of progress to end-stage disease is unpredictable, with the majority of these patients dying from multi-organ failure. Inhibiting progression in spontaneously breathing patients with mild to moderate COVID-19 could translate to reduced morbidity and mortality and in a lower use of limited healthcare resources.

The SARS-COV-2 virus may cause gastrointestinal symptoms, such as vomiting, diarrhea, or abdominal pain during the early phases of the disease. Gastrointestinal dysfunction may induce changes in intestinal microbes and an increase in inflammatory cytokines.

Mebendazole is an anthelmintic agent that exhibits broad spectrum activity against single or mixed helminthic infestations. Mebendazole binds selectively to tubulin in the intestines of helminths and interferes with microtubule formation, blocking glucose uptake and generation of ATP, resulting in impaired digestive function, inhibition of larval development, and death of the helminths. Preclinical studies suggest that Mebendazole may have anti-neoplastic activity by inhibiting a wide range of factors involved in tumor progression such as tubulin polymerization, angiogenesis, matrix metalloproteinases, and multi-drug resistance protein transporters. Mebendazole was also shown to inhibit several drug transporters including ATP-binding cassette (Imatinib) transporters.

The SARS-COV-2 virus has spike proteins that are known to interact with cytoskeleton filaments (e.g., actin, tubulin) for successful entry of the virus into host cells. Entry of the virus is a key step in viral infection.

Following oral administration of Mebendazole, peak plasma concentrations are achieved in 2 to 4 hours. Animal and human studies indicate slight to moderate oral absorption. In humans, less than about 10% of a single oral dose reaches systemic circulation due to extensive first-pass metabolism. An oral bioavailability of 17% has been reported. Following administration of 100 mg twice daily for three consecutive days, plasma concentrations of Mebendazole did not exceed 0.03 μg/mL (0.1 μM). Following long-term oral administration, increased plasma concentrations resulted in about a 3-fold higher exposure to steady-state. In subjects on chronic Mebendazole chemotherapy, peak plasma concentrations ranged from 0.1 to 0.5 μg/mL (0.3 to 1.69 μM). Systemic exposure is reportedly higher in children compared to adults. Evidence suggests small amounts of Mebendazole are present in human milk following oral administration. Mebendazole is extensively metabolized in the liver to several inactive metabolites that demonstrate higher plasma concentrations compared to those of Mebendazole. The metabolites of Mebendazole likely undergo enterohepatic recirculation. Excretion is primarily fecal; <2% of an oral dose is excreted in urine. The elimination half-life ranges from 3 to 6 hours.

Imatinib is a kinase inhibitor indicated for treatment of adult and pediatric patients with Philadelphia chromosome positive chronic myeloid leukemia (Ph+ CML) and acute lymphoblastic leukemia (Ph+ ALL).

In addition to the anti-inflammatory effects, Imatinib also exhibits antiviral properties. The antiviral activity of Imatinib appears to occur at the early stages of infection, following cellular entry and endosomal trafficking, by inhibiting fusion of the virus at the endosomal membrane. The potential antiviral properties of this drug have been previously demonstrated in preclinical assays where Imatinib demonstrated a potent inhibitory effect on SARS-COV and MERS-COV replication in in vitro assays, mainly via its inhibition of ABL type 2 kinase. In these studies, Imatinib demonstrated low toxicity and inhibited SARS-COV and MERS-COV with EC50 values in the range of 9.8 to 17.6 μM. These data suggest that the ABL1 pathway may be important for replication of different virus families and, therefore, inhibitors of this pathway have the potential to be broad-spectrum antivirals. Additionally, pharmacokinetic studies have demonstrated that the IC50 of Imatinib for ABL1, BCR-ABL1, and ABL2 kinase inhibition is around 0.3 μM, which is below the expected trough plasma concentration (1.7 μM) of an oral Imatinib dose of 400 mg/day. Initial estimates suggest an EC50 value of approximately 2.5 μM for the inhibition of SARS-COV-2 virus. This concentration is achievable in vivo following administration of an oral dose of about 800 mg/day Imatinib in patients, thus one of the therapeutically effective amounts comprised in the combination therapy according to embodiments of the present disclosure.

Based on pre-clinical models, Mebendazole given orally, showed only about 15% bioavailability and the rest of the drug remained in the gastrointestinal tract. On the contrary, Imatinib showed an excellent absorption (98% bioavailability after oral administration). In some embodiments of the present disclosure, the two drugs may be given with at least 60-minutes between administration. For example, treatment could start with administering a therapeutically effective amount of Imatinib, waiting about 60 minutes or more, followed by administering a therapeutically effective amount of Mebendazole. This approach could minimize any possible drug-drug interactions during treatment.

In some embodiments of the present disclosure, Mebendazole and Imatinib may be used as agents, individually or in combination, with the same or different therapeutically effective amounts. As a non-limiting example, Mebendazole and Imatinib may be provided in one or two or more medicaments that deliver a dose of between 1 mg and 1000 mg of each or both of Mebendazole and Imatinib. In further non-limiting examples, the one or two or more medicaments may deliver a single dose of each or both of Mebendazole and Imatinib of between about 5 mg and about 995 mg, between about 10 mg and about 990 mg, between about 25 mg and about 975 mg, between about 50 mg and 950 mg, between about 75 mg and 925 mg, between about 100 mg and about 900 mg, between about 200 mg and 800 mg, between about 300 mg and 700 mg, between about 500 mg and 600 mg and combinations thereof.

Some embodiments of the present disclosure relate to a pharmaceutical composition that comprises both Mebendazole and Imatinib with the same or different therapeutically effective amounts of each agent for treating COVID-19. In some embodiments of the present disclosure, the pharmaceutical composition further comprises one or more carriers and/or one or more excipients.

### Example 1-Protein-Protein Binding Modelling

The Louisiana State University (LSU) DeepDrug™ computational artificial intelligence (AI) system identified Mebendazole and Imatinib as likely to be effective against SARS-COV-2 based on the similarity of the drugs to antiviral peptides (AVPs) known to target SARS-COV-1 and other viruses. AVPs are fragments of human proteins that respond to a viral infection by targeting key steps in the viral replication life-cycle including (1) virus binding to the cell surface and internalizing into endosomal compartments (entry), (2) virus being released from endosomal compartments into the cytosol (fusion), and (3) viral protein processing and replication of the viral genome (replication).

An AI technique was used to generate “fingerprints” for Mebendazole, Imatinib, and the AVPs in a mathematical representation of all protein interactions in a cell. This mathematical representation was created based on the following datasets: AVPdb, a dataset of 2,683 AVPs including 98 from SARS-CoV-1; HPIDB, a dataset of 981 HIV AVPs; hu.map, a dataset of 17.5 million protein-protein interactions; Corum, a dataset of 4,274 mammalian protein complexes; STRING, a dataset of 4,584,628 proteins from 5,090 organisms; DrugBank, a dataset of 13,491 drugs; and BindingDB, a dataset of 846,857 drugs and 7,605 protein targets.

An AI technique called a Siamese Network (SNet) was used to compare the fingerprints of Mebendazole and Imatinib to the fingerprints of AVPs. SNet predictions were based on a small number of SARS-COV-1 AVPs that exhibited the strongest antiviral effects. Additionally, SNet provided separate predictions for the three mechanisms of viral infection (e.g., entry, fusion, and replication), which afforded a higher degree of specificity in drug screening. The SNet projected fingerprints into a multidimensional space and calculated distances between them, and the closer the prediction was to zero, the more similar a pair of fingerprints were and the more a drug resembled AVPs. Predictions less than the optimal threshold of 0.63 indicate a significant similarity between the fingerprint of a drug and AVP (i.e., a drug having antiviral effects).

The three mechanisms are relevant for the following reasons: entry is important because inhibiting viral entry into the cell would reduce the amount of virus that acts on a subject cell.

Fusion is worth noting because not all viral entry into a subject cell occurs through the standard mechanisms. In some instances, the virus may be capable of fusing directly with the membrane of the subject cell and through this fusion, the virus can enter and infect the subject cell. Though this happens at about 1/10th the rate of the standard entry mechanism, it is still a mechanism which was desirable to target for inhibiting.

Likewise, inhibition of replication is important for reducing the amount of viral load generated and spread to other cells after a cell has been infected. Finally, the fingerprints of these specific peptides were created by using the human proteome and a large graph of the proteins involved in all the processes therein. By comparing these fingerprints to the drug fingerprints, the identification of drugs with a potential for a similar (antiviral) effect on the human proteome as the AVPs was carried out.

Both Imatinib and Mebendazole received significant support (i.e., SNet distances closest to zero) for each of the viral mechanisms (e.g., entry, fusion, and replication). Based on comprehensive analysis of SNet predictions for 4,118 FDA-approved drugs, Imatinib and Mebendazole ranked within the top 99th percentile for each mechanism (Table 1). Although several other tyrosine kinase inhibitors and antiparasitic drugs were identified as having SNet distances close to zero, Imatinib and Mebendazole are currently off-patent (generic) FDA-approved drugs indicated for treatment of human diseases.

TABLE 1
SNet Predictions of Imatinib and Mebendazole
for Entry, Fusion and Replication

| | Entry | Fusion | Replication |
|---|---|---|---|
| | Prediction | Prediction | Prediction |
| | [Ranking] | [Ranking] | [Ranking] |
| Drug | (Percentile) | (Percentile) | (Percentile) |
| Imatinib | 0.1455 | 0.0955 | 0.2424 |
| | [20^{th}] | [9^{th}] | [27^{th}] |
| | (99.51) | (99.78) | (99.34) |
| Mebendazole | 0.1072 | 0.0796 | 0.2033 |
| | [4^{th}] | [2^{nd}] | [10^{th}] |
| | (99.90) | (99.95) | (99.76) |

An assessment of the potential of small therapeutics to bind with COVID-19 virus particles was carried out. Using three different mechanisms for potential binding sites for small molecules, the likelihood of protein-protein binding was determined. Using a template of the crystal structure of an essential SARS-COV-2 protease, the functional centers of the protease inhibitor-binding pocket were identified.

### First Binding Mechanism

A number of therapeutic compounds were studied to determine their propensity to bind to COVID-19 particles according to a first binding mechanism. The interactions where further evaluated by assessing the likelihood the therapeutic compounds would impact the entry of COVID-19 into mammalian cells; the fusion of COVID-19 particles with mammalian cells; and ultimately the replication of the COVID-19 infected cells. Table 1 summarizes the data obtained in this first round of modeling data analysis.

TABLE 2
Results of Protein-Protein modeling data which mimics
a first mechanism of interaction between COVID-19
and each one of the identified compounds

| | Modeling scores (higher than 0.25 = favorable score) | | | | |
|---|---|---|---|---|---|
| | | | | | Entry, Fusion |
| Drug | Corona | Entry | Fusion | Replication | or replication |
| Mebendazole | 0.6323 | 0.3207 | 0.3885 | 0.0847 | Entry & Fusion |
| Imatinib | 0.5696 | 0.0892 | 0.0092 | 0.3522 | Replication |

According to the data collected in the study of the first binding mechanism, the tested compounds demonstrated a propensity to bind to COVID-19 particles.

### Second Binding Mechanism

The same compounds were subsequently studied to determine their propensity to bind to COVID-19 particles according to a second binding mechanism. The interactions where also further evaluated by assessing the likelihood the therapeutic compounds would impact the entry of COVID-19 into mammalian cells; the fusion of COVID-19 particles with mammalian cells; and ultimately the replication of the COVID-19 infected cells. Table 2 summarizes the data obtained in this second round of modeling data analysis.

TABLE 3
Results of Protein-Protein modeling data which mimics
a second mechanism of interaction between COVID-19
and each one of the identified compounds

| | Modeling scores (lower than 0.5 = unfavorable score) | | |
|---|---|---|---|
| Drug | Entry | Fusion | Replication |
| Mebendazole | 0.8928 | 0.9204 | 0.7967 |
| Imatinib | 0.8545 | 0.9045 | 0.7576 |

According to the data collected in the study of the second binding mechanism, the tested compounds demonstrated a propensity to bind to COVID-19 particles.

### Third Binding Mechanism

The same compounds were again studied to determine their propensity to bind to COVID-19 particles according to a third binding mechanism. The interactions where also further evaluated by assessing the likelihood the therapeutic compounds would impact the entry of COVID-19 into mammalian cells; the fusion of COVID-19 particles with mammalian cells; and ultimately the replication of the COVID-19 infected cells. Table 3 summarizes the data obtained in this third round of modeling data analysis.

TABLE 3
Results of Protein-Protein modeling data which mimics
a third mechanism of interaction between COVID-19
and each one of the identified compounds

| | Modeling scores Cos Sim (higher = better) | | |
|---|---|---|---|
| Drug | Entry | Fusion | Replication |
| Mebendazole | 0.725615118 | 0.719017032 | 0.645826892 |
| Imatinib | 0.527941877 | 0.61502544 | 0.486479492 |

According to the data collected in the study of the third binding mechanism, the tested compounds demonstrated a propensity to bind to COVID-19 particles.

Using data from the public domain, a causality analysis was performed to identify proteins directly or indirectly affected by Mebendazole. For Mebendazole, protein interaction scores for 22 proteins were identified: the top four were calmodulin-domain protein kinase 1 (CDPK1) from the parasite Toxoplasma gondii (a score of 0.67), vascular endothelial growth factor receptor 2 (VEGF2) (0.62), Abelson tyrosine protein kinase 1 (ABL1) (0.57), and proto-oncogene tyrosine protein kinase Src (SRC) (0.55) (Table 4). Several viral proteins were identified as potential protein targets of Mebendazole, including genome polyprotein from West Nile virus (0.20), RNA polymerase subunit P3 from influenza A virus (0.15), and capsid protein from Hepatitis B virus. These data support a direct or indirect interaction between Mebendazole and viral proteins that are involved in the processing, transport, and replication of viruses. Although viral proteins of coronaviruses were not identified using this analytical method, which could have been due to a limited availability of data, other analyses indicated a potential for Mebendazole to have antiviral effects similar to antiviral peptides that target SARS-COV-1.

TABLE 4
Protein Interaction Scores Derived from Causality Analysis for Mebendazole

| Protein Interaction | | | |
|---|---|---|---|
| Score for Mebendazole | Protein Name | Organism | UniProt ID |
| 0.67 | Calmodulin-domain protein kinase 1 | Toxoplasma gondii | Q9BJF5 |
| 0.62 | Vascular endothelial growth factor receptor 2 | Human | P35968 |
| 0.57 | Abelson tyrosine-protein kinase 1 | Human | P00519 |
| 0.55 | Proto-oncogene tyrosine-protein kinase Src | Human | P12931 |
| 0.43 | Integrin alpha-5 | Human | P06756 |
| 0.40 | Adenylate cyclase type 6 | Human | O43306 |
| 0.20 | Genome polyprotein | West Nile Virus | P06935 |
| 0.16 | Lysosomal-associated membrane protein 3 | Human | P08962 |
| 0.15 | Trans-activator protein BZLF1 | Epstein-Barr Virus | P03206 |
| 0.15 | RNA-directed RNA polymerase subunit P3 | Influenza A Virus | P03428 |
| 0.11 | Capsid protein | Hepatitis B Virus | Q76R61 |
| 0.09 | Integrin alpha-3 | Human | P26006 |
| 0.09 | Tyrosine-protein kinase Yes | Human | P07947 |
| 0.08 | Protein LANA1 | Herpes Virus | Q9QR71 |
| 0.08 | Signal Peptide peptidase-like 2A | Human | Q8TCT8 |
| 0.07 | Tyrosine-protein kinase Lyn | Human | P07948 |
| 0.02 | Tyrosine-protein kinase Fyn | Human | P06241 |
| 0.02 | Insulin-like growth factor 1 receptor | Human | P08069 |
| 0.02 | Protein kinase C Alpha type | Human | P17252 |
| 0.02 | Insulin receptor | Human | P06213 |
| 0.02 | Ephrin type-A receptor 2 | Human | P29317 |
| 0.01 | Casein kinase I isoform gamma-3 | Human | Q9Y6M4 |

A similar analysis was also identified for Imatinib, which produced protein interaction scores for 36 proteins, including a number of tyrosine protein kinases (Table 4B).

TABLE 4B
Protein Interaction Scores Derived from Causality Analysis for Imatinib

| Protein Interaction | | | |
|---|---|---|---|
| Score for Imatinib | Protein Name | Organism | UniProt ID |
| 0.83 | Serine/threonine-protein kinase Chk1 | Human | O14757 |
| 0.83 | Protein kinase C eta type (PKC-L) | Human | P24723 |
| 0.83 | Myotonin-protein kinase | Human | Q09013 |
| 0.83 | cGMP-dependent protein kinase 1 | Human | Q13976 |
| 0.83 | Aurora kinase B | Human | Q96GD4 |
| 0.83 | Serine/threonine-protein kinase D2 | Human | Q9BZL6 |
| 0.83 | Interleukin-1 receptor-associated kinase 3 | Human | Q9Y616 |
| 0.80 | Platelet-derived growth factor receptor beta | Human | P09619 |
| 0.80 | Discoidin domain-containing receptor 2 | Human | Q16832 |
| 0.79 | Platelet-derived growth factor receptor alpha | Human | P16234 |
| 0.76 | Breakpoint cluster region protein | Human | P11274 |
| 0.75 | Phosphatidylinositol 5-phosphate 4-kinase type-2 gamma | Human | Q8TBX8 |
| 0.75 | Tyorsine-protein kinase Blk | Human | P51451 |
| 0.73 | Homeodomain-interacting protein kinase 4 | Human | Q8NE63 |
| 0.73 | Cyclin-G-associated kinase | Human | O14976 |
| 0.73 | Interleukin-1 receptor-associated kinase 1 | Human | P51617 |
| 0.72 | Ephrin type-A receptor 8 | Human | P29322 |
| 0.72 | Tyrosine-protein kinase FRK | Human | P42685 |
| 0.72 | Platelet-derived growth factor receptor beta | Rat | Q05030 |
| 0.72 | RAF proto-oncogene serine/threonine-protein kinase | Human | P04049 |
| 0.72 | Maternal embryonic leucine zipper kinase | Human | Q14680 |
| 0.71 | Dual specificity protein kinase CLK4 | Human | Q9HAZ1 |
| 0.71 | Tyrosine-protein kinase Fgr | Human | P09769 |
| 0.71 | Mitogen-activated protein kinase kinase kinase 20 | Human | Q9NYL2 |
| 0.70 | Tyrosine-protein kinase Fyn | Human | P06241 |
| 0.70 | Mitogen-activated protein kinase 10 | Human | P53779 |
| 0.70 | Serine/threonine-protein kinase B-raf | Human | P15056 |
| 0.70 | Serine/threonine-protein kinase TNNI3K | Human | Q59H18 |
| 0.70 | Dual specificity protein kinase CLK1 | Human | P49759 |
| 0.69 | Mitogen-activated protein kinase 8 | Human | P45983 |
| 0.69 | Serine/threonine-protein kinase 17 | Human | Q9UEE5 |
| 0.69 | Serine/threonine-protein kinase PLK4 | Human | O00444 |
| 0.64 | Inner centromere protein | Human | Q9NQS7 |
| 0.61 | Vascular endothelial growth factor receptor 2 | Human | P35968 |
| 0.61 | Abelson tyrosine-protein kinase 1 | Human | P00519 |
| 0.61 | Proto-oncogene tyrosine-protein kinase Src | Human | P12931 |

### Example 2: In Vitro Studies

The in vitro efficacy of Mebendazole and other potential antiviral compounds against SARS-COV-2 (USA-WA1/2020 Isolate) was assessed in human lung cancer (Calu-3) cells. A stock solution of 4 μM of Mebendazole was prepared in DMSO and tested at eight concentrations of 5000 nM, 1670 nM, 555.6 nM, 185.2 nM, 61.7 nM, 20.6 nM, 6.9 nM, and 2.3 nM. Calu-3 cells were cultured in 96-well plates and tested in triplicate. A pretreatment/treatment regimen was utilized where cells were incubated with Mebendazole for 24±4 hours, cells were then inoculated at a multiplicity of infection (MOI) of 0.005 TCID₅₀ (median tissue culture infectious dose) per cell (200 TCID₅₀/well) with SARS-COV-2 and incubated for 60-90 minutes. Immediately following incubation, viral inoculum was removed, cells were washed, and wells were overlaid with 0.2 mL Eagle's Modified Essential Media (EMEM) with 2% Fetal Bovine Serum (FBS) containing Mebendazole or control articles and incubated in a humidified chamber at 37° C.±2° C. in 5±2% CO₂. At 48±6 hours following virus inoculation, cells were fixed and evaluated for the presence of virus by the immunostaining assay (for cytopathic effect (CPE) by the MTT [3-(4,5-dimethylthiazol-2-yl)-2,5-diphenyltetrazolium bromide] assay).

For each well on the assay plate, inhibition of SARS-COV-2 was calculated as the percentage of reduction of the absorbance value (A450 of Mebendazole dilution) relative to mean absorbance values from wells designated as positive control (A450 virus control; no drug and 0% inhibition) and negative control (A450 of cell control; no virus and 100% inhibition) by the following formula:

$$Percent Inhibition = 100 - ( \frac{A 450 of mebendazole dilution - A 450 of cell control}{A 450 of virus control - A 450 of cell control} ) \times 100$$

The effective concentration (EC₅₀) was defined as the concentration of Mebendazole that causes 50% reduction of the mean absorbance value of the virus control (0% inhibition) relative to the cell control (100% inhibition). The cytotoxicity of Mebendazole at the 8 concentrations was also evaluated as determined by percent inhibition of cell viability.

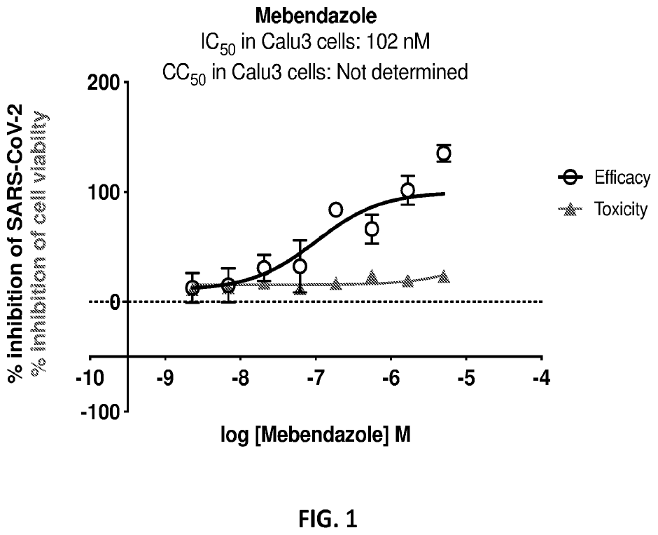

FIG. 1 shows concentration-response curves for the efficacy (% inhibition of SARS-COV-2) and cytotoxicity (% inhibition of cell viability) of Mebendazole at 2.3 nM-5000 nM. Calu-3 cells were infected with SARS-COV-2 at an MOI of 0.005 TCID50/cell (200 TCID50/well). Mebendazole was shown to be active against SARS-COV-2 (with an EC₅₀ of 102 nM) and with no apparent cytotoxicity (CC50>5000 nM). Inhibition of 100% of SARS-COV-2 with an EC₅₀ of 102 nM was determined for Mebendazole. The cytotoxic concentration (CC₅₀) was estimated at >5 μM based on an absence of observed cytotoxicity (˜0% inhibition of cell viability) at all tested concentrations up to a maximum concentration of 5000 nM, indicating a wide margin of safety between the effective concentrations and the cytotoxic concentrations reported in these human lung cells.

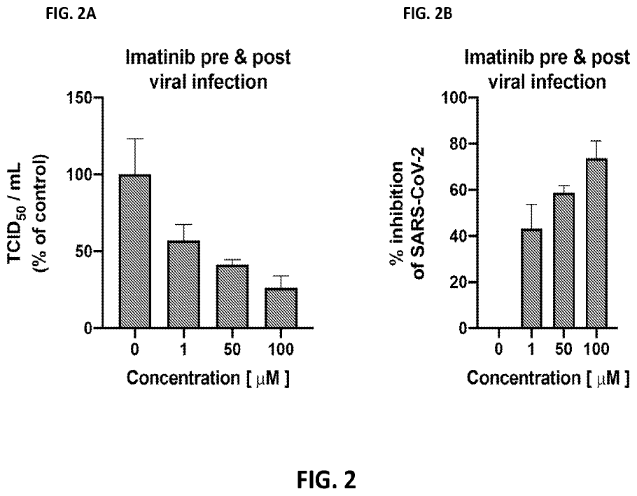

Further in vitro experiments demonstrated that Imatinib also inhibited SARS-COV-2 in Vero 76 African green monkey kidney cells infected with SARS-COV-2 (New York-PV091158/2020 strain) Imatinib was added to cells at the same time as the virus (“Imatinib pre & post viral infection”), or after a 1-hour incubation with the virus (“Imatinib post viral infection”) in order to evaluate if the presence of Imatinib could impair entry of the virus into the cell, which could be represented in the data as increased SARS-COV-2 inhibition. For the pre-& post-treatment arm, percent inhibition of SARS-COV-2 by Imatinib was 43% (1 μM), 51% (50 μM), and 74% (100 μM) (see FIG. 2B). For the post-treatment arm, SARS-CoV-2 inhibition by Imatinib was 24% (1 μM), 3% (50 μM), and 55% (100 μM) (data not shown). For both arms, SARS-COV-2 inhibition by the highest concentration of Imatinib (100 μM) was statistically significant by one-way ANOVA analysis.

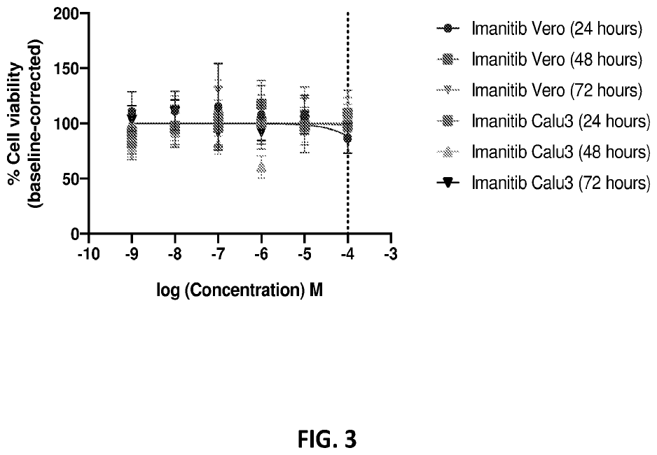

Cytotoxicity experiments were performed in parallel using the same experimental conditions and including another cell line (Calu-3) in addition to Vero 76 cells, and no cytotoxicity was observed at any concentration tested (up to 100 μM) (see FIG. 3). These data show that Imatinib appreciably inhibited SARS-COV-2 at concentrations as low as 1 μM and was not cytotoxic at 100 μM, lending support for a good therapeutic index in humans. Furthermore, increased antiviral activity was observed when Imatinib was added to cells prior to the addition of the virus to the cells, suggesting that Imatinib may be affecting how the virus enters cells in addition to affecting viral replication.

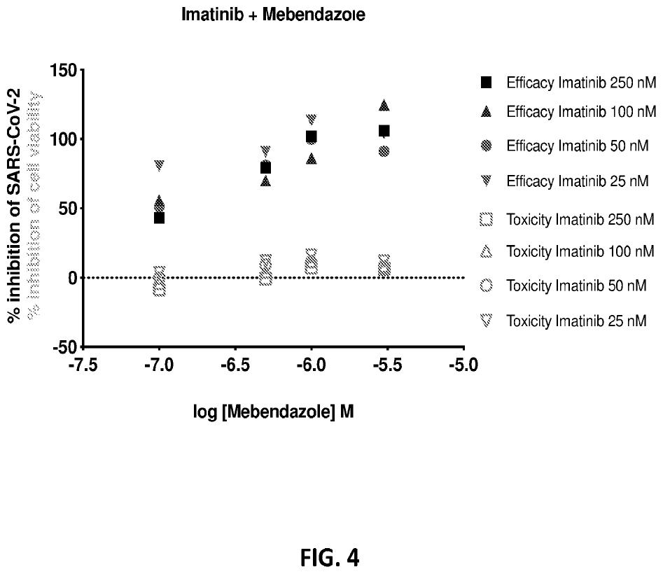

The in vitro efficacy and cytotoxicity of Imatinib in combination with Mebendazole was evaluated in Calu-3 cells (see FIG. 4). Imatinib at 4 different concentrations (25, 50, 100, or 250 nM) and Mebendazole at 4 concentrations (0.1, 0.3, 1, and 3 μM) were added to cells following the same protocol as described above for FIG. 1. Imatinib in combination with Mebendazole at these drug concentrations achieved dose-dependent inhibition of SARS-COV-2 without evidence of cytotoxicity.

### Example 3: In Vivo Study

The objective of the in vivo study was to test the in vivo efficacy of antiviral therapeutics against SARS-COV-2, the causative agent of COVID-19, in the ACE2 mouse model, using female mice (strain: B6.Cg-Tg (K18-hACE2) 2Prlmn/J). Efficacy was determined by quantifying viral shedding, as measured by RT-qPCR, and viral infectivity, as measured by a TCID50 analysis of lung tissue, both of which are standard endpoints to quantify the amount of infectious virus in animal models of viral infection.

In brief, the study protocol was as follows: At initiation of dosing (Day 0), mice were approximately 7-10 weeks old, weighed between 15.7-22.3 g, and were quarantined for at least seven days prior to assignment to the study. Mice were anesthetized via an intraperitoneal (IP) injection of ketamine (100 mg/kg) and xylazine (10 mg/kg) mixture and challenged intranasally with a 30 μL solution containing 5×10³ TCID₅₀ SARS-COV-2 virus (USA WA1/2020). Following exposure to the inoculum the mouse was held upright to allow the virus to be inhaled thoroughly then the mouse was returned to its cage. A quantitative viral infectivity assay (e.g., TCID50 assay) was performed at on a portion of the prepared viral challenge solution. Mice (11 in each group) were administered virus 4-6 hours prior to administration of placebo, 50 mg/kg/day Mebendazole, 100 mg/kg/day Imatinib or 50 mg/kg/day Mebendazole and 100 mg/kg/day Imatinib, once daily for 10 days by oral gavage. All handling of SARS-COV-2 virus was performed under Biosafety Level 3 conditions.

Animal rooms were lighted with fluorescent lights and maintained on a 12-hr light/dark cycle. To the maximum extent possible, room temperature was maintained at approximately 20-26° C. and relative humidity at approximately 30-70% in accordance with the National Research Council's “Guide for the Care and Use of Laboratory Animals”, 2011. Room temperature and relative humidity values were recorded daily.

Throughout the study, mice were observed twice daily for mortality or evidence of moribundity, and abnormal clinical signs were recorded. On Day 6, the interim endpoint, a gross necropsy examination was performed on the lungs and the left lung was harvested for RT-qPCR and TCID₅₀ analysis. Additionally, nasal turbinate, gastrointestinal tissue (e.g., stomach, jejunum, ileum, and colon), leg bone, and bronchial lymph nodes were collected for microscopic examination and blood was collected for clinical chemistry determinations.

The concentration of virus in lung tissue samples was determined by RT-qPCR analysis. RNA was extracted using the Quick-RNA Viral Kit (Zymo Research) according to the manufacturer's protocol and stored in RNA/DNA Shield (Zymo Research). RT-qPCR analysis was performed using the iTaq Universal Probes One-Step Kit (Bio-Rad). The following RT-qPCR cycling conditions were used: 50° C. for 15 minutes (reverse transcription), 95° C. for 2 minutes (denature), then 40 cycles of 10 seconds at 95° C. followed by 45 seconds at 62° C. The concentration of the virus in the lung was determined using RT-qPCR using the primers indicated in Table 5 below.

TABLE 5
RT-qPCR Primer Sequences

| Primer | Sequence |
|---|---|
| 2019-nCOV_N1-F | 5′-GACCCCAAAATCAGCGAAA |
| | T-3′ |
| 2019-nCOV_N1-R | 5′-TCTGGTTACTGCCAGTTGA |
| | ATCTG-3′ |
| Probe: 2019-nCOV_N1-P | 5′-FAM-ACCCCGCATTACGTT |
| | TGGTGGACC-BHQ1-3′ |

RT-qPCR analysis of lung samples (viral titer/mRNA levels) from treated mice are shown in Table 6 and Table 7. The mean viral titer of the Mebendazole-treated mice and Mebendazole+Imatinib-treated mice was lower compared to placebo, with a percent reduction of 44.2% and 42.4%, respectively, when compared to untreated control. However, no statistically significant differences were observed using a one-way analysis of variance (ANOVA) with a Tukey's post-hoc comparison. Though differences were not statistically significant between group means, there was a biologically significant trend towards reduced viral titers in mice treated with Mebendazole alone and Mebendazole+Imatinib. For example, two mice treated with Mebendazole+Imatinib (animals #62 and #63) showed a ˜97% decrease in viral titer compared to the group mean of placebo-treated mice.

TABLE 6
Lung Tissue RT-qPCR Titers

| | Animal | | | | Animal | | |
|---|---|---|---|---|---|---|---|
| Treatment | Number | Viral Titer | Log₁₀ | Treatment | Number | Viral Titer | Log₁₀ |
| Placebo | 01 | 4.86E+07 | 7.7 | Imatinib | 13 | 7.53E+07 | 7.9 |
| | 02 | 6.40E+07 | 7.8 | | 14 | 3.12E+08 | 8.5 |
| | 03 | 6.67E+07 | 7.8 | | 15 | 3.67E+07 | 7.6 |
| | 04 | 2.54E+06 | 6.4 | | 16 | 5.37E+07 | 7.7 |
| | 05 | 8.70E+07 | 7.9 | | 17 | 7.80E+07 | 7.9 |
| | 06 | 2.13E+08 | 8.3 | | 18 | 9.13E+07 | 8.0 |
| | 07 | 6.51E+07 | 7.8 | | 19 | 2.15E+07 | 7.3 |
| | Group | 7.81E+07 | 7.7 | | Group | 9.54E+07 | 7.8 |
| | Mean | (6.50E+07) | (0.59) | | Mean | (9.86E+07) | (0.37) |
| | (S.D.) | | | | (S.D.) | | |
| Mebendazole | 45 | 7.15E+07 | 7.9 | Imatinib + | 56 | 2.41E+07 | 7.4 |
| | 48 | 4.97E+07 | 7.7 | Mebendazole | 58 | 1.94E+07 | 7.3 |
| | 49 | 7.76E+07 | 7.9 | | 59 | 1.51E+08 | 8.2 |
| | 50 | 1.90E+07 | 7.3 | | 60 | 3.92E+07 | 7.6 |
| | 52 | 2.03E+07 | 7.3 | | 61 | 7.66E+07 | 7.9 |
| | 54 | 3.73E+07 | 7.6 | | 62 | 2.36E+06 | 6.4 |
| | 55 | 2.95E+07 | 7.5 | | 63 | 2.11E+06 | 6.3 |
| | Group | 4.36E+07 | 7.6 | | Group | 4.50E+07 | 7.3 |
| | Mean | (2.37E+07) | (0.25) | | Mean | (5.33E+07) | (0.72) |
| | (S.D.) | | | | (S.D.) | | |

TABLE 7
Percent Reduction of Virus in Lung Samples

| Animal Number | |
|---|---|
| | Percent Reduction of Lung Tissue RT-qPCR |
| | Titers from Mebendazole Treated Mice |
| | (Individual Animals) vs. the Group Mean of |
| | Placebo Treated Mice |
| 45 | 8.45% |
| 48 | 36.36% |
| 49 | 0.64% |
| 50 | 75.67% |
| 52 | 74.01% |
| 54 | 52.24% |
| 55 | 62.23% |
| Mean % Reduction | 44.2% |
| | Percent Reduction of Lung Tissue RT-qPCR |
| | Titers from Mebendazole + Imatinib Treated |
| | Mice (Individual Animals) vs. the Group Mean |
| | of Placebo Treated Mice |
| 56 | 69.1% |
| 58 | 75.2% |
| 59 | ND |
| 60 | 49.8% |
| 61 | 1.90% |
| 62 | 97.0% |
| 63 | 97.3% |
| Mean % Reduction | 42.4% |

ND Not Determined because the viral titer of this animal was greater than the viral titer group mean of placebo treated mice.

The results of the TCID₅₀ analysis of lung samples are shown in Table 8. Briefly, Vero C1008 (E6) cells were inoculated with 100 μL of appropriate dilution of virus stock or treated with lung tissue samples and incubated at 37° C.±2° C., 5%±1% CO₂, >70% relative humidity for 120 hours. After 120 hours plates were removed from the incubator and scored for the presence of cytopathic effects (CPE) using an inverted microscope. Although differences between groups were also statistically non-significant, there was a trend towards decreased titers in the Mebendazole treated mice and Mebendazole+Imatinib-treated mice. In the TCID₅₀ assay, decreased viral titer was observed for 5 out of 7 Mebendazole+Imatinib treated mice, but this was not the case for Imatinib-treated mice, demonstrating a potential for Mebendazole to have more direct antiviral effects against SARS-COV-2. Based on the mechanism of action, Imatinib is suggested to reduce the pathological effects of viral infection (i.e., reducing the severity of symptoms by acting as an anti-inflammatory agent and reducing cytokine storm).

TABLE 8
TCID₅₀ Analysis Samples (RT-qPCR data)

| | | Titer | | | Titer |
|---|---|---|---|---|---|
| | | (Log₁₀ | | | (Log₁₀ |
| | Animal | TCID₅₀/ | | Animal | TCID₅₀/ |
| Treatment | Number | mL) | Treatment | Number | mL) |
| Placebo | 01 | 6.5 | Imatinib | 13 | 7.6 |
| | 02 | 6.5 | | 14 | 8.5 |
| | 03 | 6.8 | | 15 | 8.0 |
| | 04 | 4.8 | | 16 | 6.5 |
| | 05 | 6.5 | | 17 | 8.3 |
| | 06 | 7.5 | | 18 | 7.5 |
| | 07 | 6.3 | | 19 | 5.3 |
| | Group | 6.4 | | Group | 7.4 |
| | Mean | (0.8) | | Mean | (1.1) |
| | (S.D.) | | | (S.D.) | |
| Mebendazole | 45 | 6.0 | Imatinib + | 56 | 5.3 |
| | 48 | 5.3 | Mebendazole | 58 | 7.3 |
| | 49 | 4.8 | | 59 | 4.8 |
| | 50 | 5.5 | | 60 | 5.8 |
| | 52 | 5.3 | | 61 | 8.5 |
| | 54 | 7.3 | | 62 | 4.3 |
| | 55 | 6.5 | | 63 | 4.5 |
| | Group | 5.8 | | Group | 5.8 |
| | Mean | (0.9) | | Mean | (1.6) |
| | (S.D.) | | | (S.D.) | |

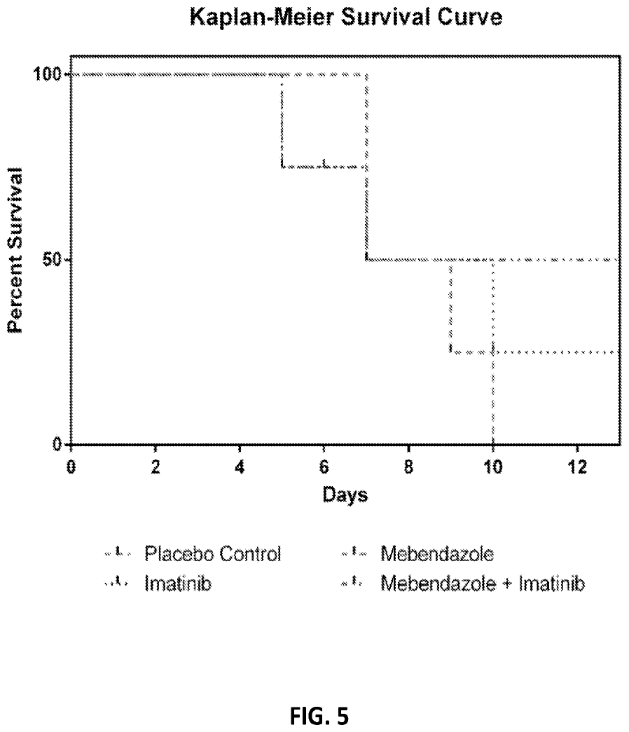

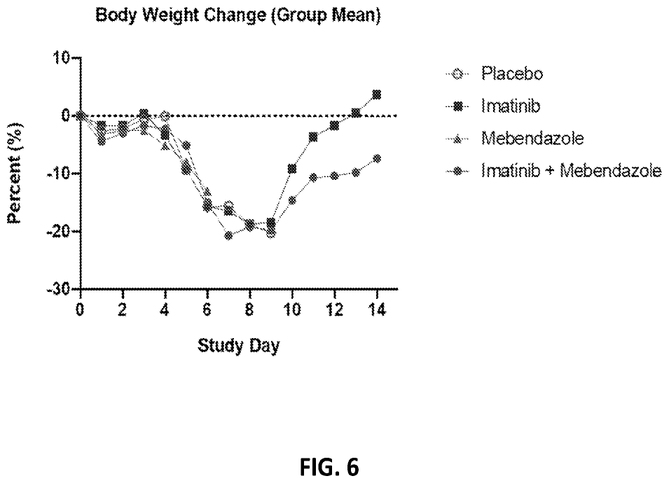

Body weights were recorded within two days of receipt and at randomization. All study animals were weighed prior to challenge then for 14 days post challenge. The change in percent change body weight was calculated for each animal. FIG. 5 shows the percent survival from study day 0 to study day 14. FIG. 6 shows the body weight data (% of initial body weight) for all of the experimental groups. Without being bound by any particular theory, it is noted that mice in experimental group treated with Mebendazole and Imatinib recovered body weight after the seven day mark.

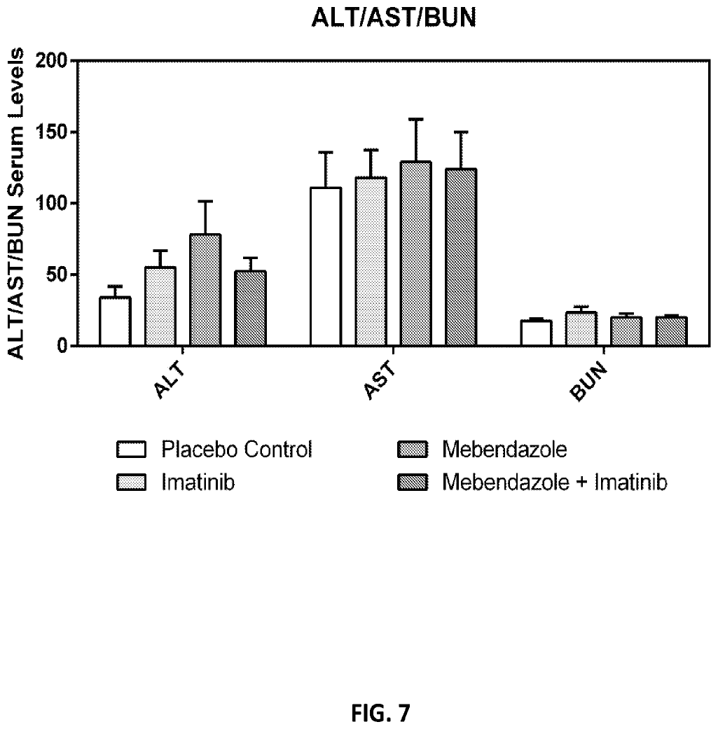

On Day 6, serum biochemistry markers were assessed for liver or renal injury and no significant changes in ALT, AST, or BUN levels were observed for Imatinib-treated, Mebendazole-treated, and Imatinib+Mebendazole-treated mice, suggesting no significant toxicities following the treatments (see FIG. 7). Furthermore, no abnormal histopathological findings in tissues and samples collected (nasal turbinates, stomach, jejunum, ileum, colon, femur, bronchial lymph node, and right lung) at necropsy were observed, supporting a favorable safety profile of Mebendazole and Imatinib in combination.

Without being bound by any particular theory, the embodiments of the present disclosure relate to treatments for subjects infected with (or likely to be infected with) one or more variants of the SARS-COV-2 virus. In some embodiments of the present disclosure, Mebendazole is administered and in some embodiments of the present disclosure, Mebendazole is administered with Imatinib. Some embodiments of the present disclosure relate to methods of preventing entry of the virus into target cells that have formed an agent/target cell complex. Some embodiments of the present disclosure relate to a method of inhibiting fusion of the virus with target cells that have made an agent/target cell complex. Some embodiments of the present disclosure relate to a method of inhibiting replication of the virus within target cells that have made an agent/target cell complex. Some embodiments of the present disclosure relate to methods of preventing entry of the virus into a subject's cells where an agent/virion complex has formed. Some embodiments of the present disclosure relate to a method of inhibiting fusion of the virus with a subject's cells where an agent/virion complex has formed. Some embodiments of the present disclosure relate to a method of inhibiting replication of the virus within a subject's cells where an agent/virion complex has formed. The agent may be Mebendazole, Imatinib or both.

1. A method for treating a corona virus infection in a subject or treating a subject exposed to a coronavirus, comprising administering Mebendazole to the subject

2. The method of claim 1, further comprising administering and Imatinib to the subject.

3. The method of claim 1, wherein treating comprises inhibiting entry of a coronavirus into a subject's cell, inhibiting fusion of a coronavirus with a subject's cell, or inhibiting replication of a coronavirus within a subject's cell.

4. (canceled)

5. (canceled)

6. The method of claim 3 further comprising administering Imatinib.

7. The method of claim 1, wherein the coronavirus infection is caused by a variant of SARS-COV-2 virus.

8. The method of claim 1, wherein said method comprises the steps of:
a. providing a therapeutically effective amount of Mebendazole; and
b. wherein, in the administering step, said therapeutically effective amount of Mebendazole is administered to said individual to ameliorate the risks or symptoms associated with a coronavirus infection.

9. The method of claim 8, further comprising a step of administering a therapeutically effective amount of a tyrosine kinase inhibitor.

10. The method of claim 9, wherein the tyrosine kinase inhibitor is Imatinib.

11. The method of claim 8, wherein the coronavirus infection is caused by a SARS-CoV-2 virus variant.

12. A method of making an agent/target cell complex or an agent/target virion complex, the method comprising a step of:
administering a therapeutically effective amount of an agent to a target cell to form the agent/target cell complex, wherein the agent/target cell complex decreases the ability of a virus to enter, fuse with and/or replicate within one or more cells of a subject; or
administering a therapeutically effective amount of an agent to a virion to form the agent/target virion complex, wherein the agent/target virion complex decreases the ability of the target virion to enter, fuse with and/or replicate within one or more cells of a subject.

13. The method of claim 12, wherein the target cell is one or more of an airway epithelial cell; an alveolar epithelial cell; an olfactory epithelial cell, olfactory neural cell; a central nervous system neuron; a peripheral nervous system neuron; a gastrointestinal epithelial cells, a gastrointestinal enterocyte; a gastrointestinal glandular cell; an immune effector cell; a cardiovascular cell; and, a renal cell.

14. (canceled)

15. The method of claim 12, wherein the agent is Mebendazole, a tyrosine kinase inhibitor, or both in combination.

16. The method of claim 15, wherein the tyrosine kinase inhibitor is Imatinib.

17. The method of claim 12, wherein the virion is of a SARS-COV-2 virus variant.

18. A pharmaceutical composition comprising:
a. Mebendazole in a first therapeutically effective amount;
b. a tyrosine kinase inhibitor in a second therapeutically effective amount, wherein the first and second therapeutically effective amounts are different or not; and
C. at least one excipient.

19. The pharmaceutical composition of claim 16, wherein the tyrosine kinase inhibitor is Imatinib.

20. The method of claim 1, further comprising administering a tyrosine kinase inhibitor.

21. The method of claim 1, wherein administering the Mebendazole forms a agent/target virion complex, wherein the target virion is a SARS-COV-2 virus variant and the agent/target virion complex decreases the ability of the target virion to enter, fuse with and/or replicate within one or more cells of a subject.
